(19) United States
(12) Patent Application Publication
(10) Pub. No.: US 2005/0165282 A1
(43) Pub. Date: Jul. 28, 2005

(54) Device for retracting body skin folds

(76) Inventor: Nady E. Nady, Skillman, NJ (US)

Correspondence Address: Gregory C. Houghton, Esq.;Mathews, Collins, Shepherd & McKay, P.A., Suite 306, 100 Thanet Circle, Princeton, NJ, 08540, US

(21) Appl. No.: 10/763,931

(22) Filed: Jan. 23, 2004

(51) Int. Cl. A61B 1/32

(52) U.S. Cl. 600/206; 600/209

(57) ABSTRACT

A device useful for retracting body skin folds during diagnostic and/or surgical procedures.

### BACKGROUND OF THE INVENTION

1. Field of the Invention

The present invention relates to a device for retracting body skin folds during diagnostic and/or surgical procedures.

2. Related Art

Obesity has become an overwhelming problem in the industrialized world. Not only does obesity contribute negatively to a person's health, obesity also greatly increases the difficulty of many diagnostic and/or surgical procedures necessary for a person. In the case of the obese patient the physician may need to push the excess fat and/or skin folds out of the way and hold the fat and/or skin folds from blocking the procedure area.

Conventionally, the fat and/or skin folds are taped up to keep the fat and/or skin folds from interfering with the procedure. However, excessive amounts of tape are generally necessary and the use of tape is unnecessarily uncomfortable to the patient, either because of irritation or allergic reaction.

It is therefore desirable to provide a device to effectively retract the excess fat/and or skin folds from interfering with diagnostic and/or surgical procedures that provides comfort to the patient.

### SUMMARY OF THE INVENTION

The present invention provides a device for retracting body skin folds during diagnostic or surgical procedures that includes an elongated element that is removably mounted to a diagnostic or surgical procedure table and an elongate arm that is adapted to be slidably mounted onto the elongated element. The elongate arm has a plurality of open channels.

Also, the device includes a plurality of flexible elements that are adapted to be received on the elongate arm. The flexible elements have a plurality of raised portions that are adapted to be received by the open channels on the elongate arm and a gripping device that is connected to each of the plurality of flexible elements. The gripping devices grasp and retract body skin folds during diagnostic or surgical procedures.

### BRIEF DESCRIPTION OF THE DRAWINGS

The advantage, nature, and various additional features of the invention will appear more fully upon consideration of the illustrative embodiments now to be described in detail in connection with accompanying drawings wherein:

FIG. 1 is a schematic diagram of a device for retracting body skin folds during diagnostic and/or surgical procedures.

FIG. 2A is a schematic diagram of a gripping device connected to a flexible element adapted to be received on the device depicted in FIG. 1 in accordance with an exemplary embodiment of the invention.

FIG. 2B is a schematic diagram of a bifurcated gripping device connected to a flexible element adapted to be received on the device depicted in FIG. 1 according to an alternate embodiment of the invention.

FIG. 3 is a schematic diagram of a gripping device used to retract a body skin fold in accordance with an exemplary embodiment of the present invention.

It should be understood that the drawings are for purposes of illustrating the concept of the invention and are not necessarily to scale.

### DETAILED DESCRIPTION OF THE INVENTION

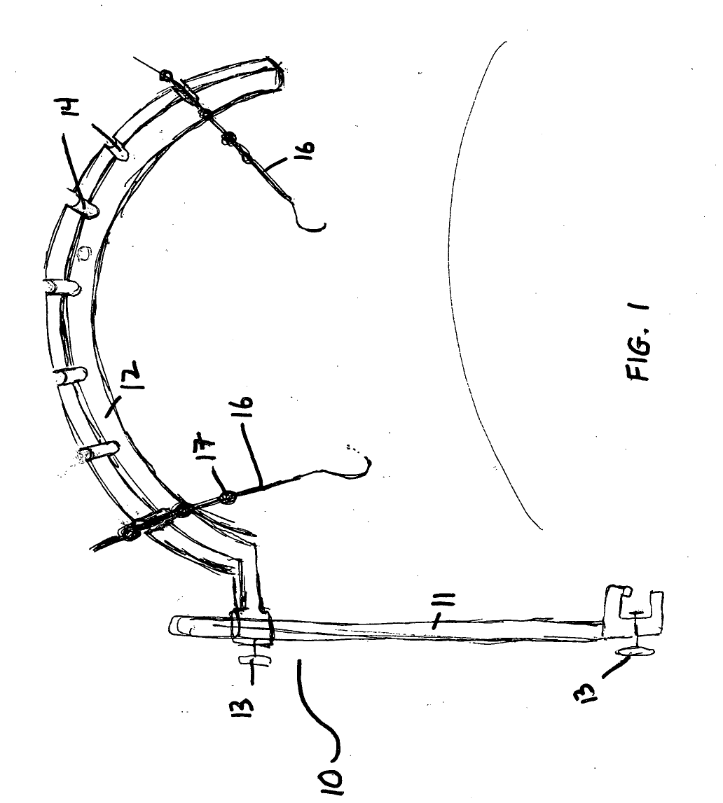

Referring to the drawings wherein like reference numerals identify similar or like elements throughout the several views and initially to FIG. 1 there is shown an exemplary embodiment of the present invention. FIG. 1 shows device 10 in accordance with the invention. Device 10 retracts body skin folds that may interfere with diagnostic and/surgical procedures.

Device 10 includes elongate element 11 and elongate arm 12. Elongate element 11 can be removably mounted to a diagnostic or surgical table (not shown) with locking device 13. Elongate element 11 can be adjusted to increase or decrease the length of elongate element 11. Locking device 13 holds elongate element 11 in a static position relative to the diagnostic or surgical table. Locking devices are well understood by those skilled in the art and include but are not limited to any device, such as clamps, hinges, and the like, that can removably mount elongate element 11 to a diagnostic and/or surgical table. Elongate arm 12 is adapted to be slidably mounted on elongate element 11. Elongate arm 12 can be adjusted up and down along elongate element 11 according to the height needed for elongate arm 12. When the desired height is determined, locking device 13 holds elongate arm 12 in a static position along elongate element 11. While elongate arm 12 is shown with a generally curved shape any shape that does not distract from the function of elongate arm 12 can be used.

Elongate arm 12 includes open channels 14. Open channels 14 are adapted to receive flexible elements 16. Flexible elements 16 include raised portions 17. Raised portions 17 hold flexible elements 16 in a temporary fixed position on elongate arm 12. Raised portions 17 limit the range of motion of flexible elements 16 horizontally through open channels 14. In an alternative embodiment raised portions 17 are spaced equally from each other according to the length of open channels 14. Raised portions 17 allow quick and easy adjustment of the length of flexible elements 16. The temporary fixed position permits a physician to quickly adjust the length of the flexible elements 16 by lifting the flexible elements 16 from open channels 14 of elongate arm 12 and either decrease or increase the suspended length of flexible elements 16 needed and place flexible elements 16 back onto the open channels 14 of elongate arm 12 with raised portions 17 keeping the flexible elements again in a temporary fixed position within open channels 14. Raised portions 17 can be any shape as understood by those skilled in the art that would hold the flexible elements in a temporary fixed position in open channels 14. In an exemplary embodiment of the present invention raised portions 17 have a generally circular area.

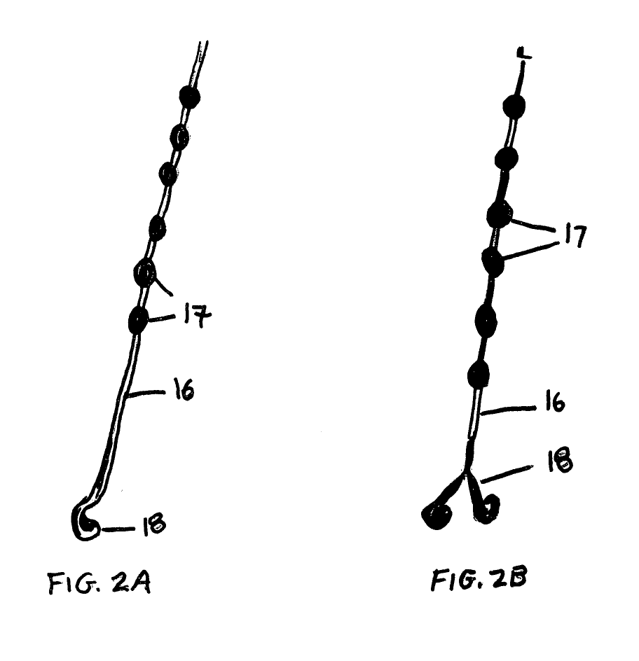

Each of flexible elements 16 include a gripping device 18, which may be bifurcated as depicted in FIG. 2B.

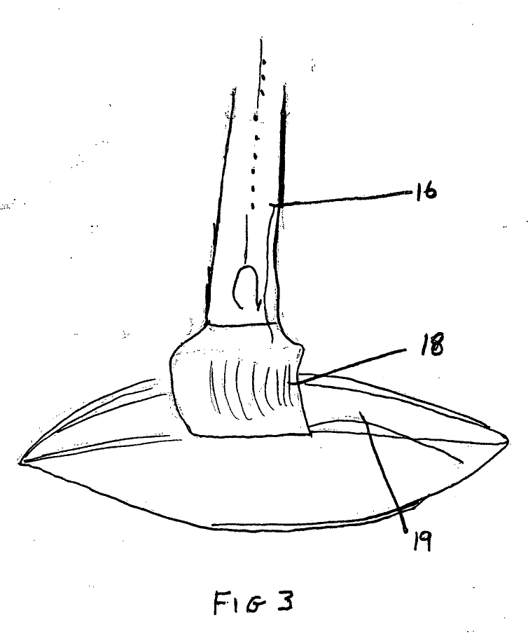

FIG. 3 schematically illustrates the use of gripping device 18 during the use of device 10. Gripping device 18 is designed to grasp and retract body skin folds 19 during diagnostic and/or surgical procedures. Gripping device 18 can be any device as understood by those skilled in the art that can grasp a body skin fold without injuring the body skin folds. The area of gripping device 18 that grasps body skin folds 19 is wide enough as to provide comfort to a patient. Gripping device 18 can be covered, such as with an elastomeric material, for example polyurethane, poly(vinyl chloride) or any other suitable material. The elastomeric material provides comfort and protects the exterior body skin folds 19. The elastomeric material can be replaceable and disposable.

Device 10 can be formed of metal or plastic.

The term body skin folds includes any body fold of a patient that may interfere with a diagnostic and/or surgical procedure. For example, body skin folds include but are not limited to fat folds and excess skin folds.

It is to be understood that the above-described embodiments are illustrative of only a few of the many possible specific embodiments which can represent applications of the principles of the invention. Numerous and varied other arrangements can be readily devised in accordance with these principles by those skilled in the art without departing from the spirit and scope of the invention.

1. A device for retracting body fat folds from interfering with diagnostic or surgical procedures comprising:
an elongated element adapted to be removably mounted to a diagnostic or surgical procedure table;
an elongate arm adapted to be slidably mounted on said elongated element, said elongate arm having a plurality of open channels;
a plurality of flexible elements adapted to be received on said elongate arm; each of said flexible elements being received in one of said open channels said flexible elements comprise a plurality of raised portions adapted to be received by said open channels to limit the range of motion of the flexible elements through the open channels; and
a gripping device connected to each of said plurality of flexible elements, where said gripping device has sufficient width to grasp and retract body fat folds from interfering with diagnostic or surgical procedures.

2. (canceled)

3. The device according to claim 1 wherein said raised portions define generally a circular area.

4. The device according to claim 1 wherein said elongate arm defines generally a curved area.

5. The device according to claim 1 wherein said gripping device is bifurcated.

6. The device according to claim 1 further comprising a material covering said gripping device.

7. The device according to claim 6 wherein said material is made of an elastomeric material.

8. The material according to claim 6 wherein said material is replaceable.

9. A method for retracting body skin folds during diagnostic or surgical procedures, the method comprising the steps of:
positioning a device over the body skin folds, said body skin folds being fat folds or excess skin folds, said device comprising an elongated element adapted to be removably mounted to a diagnostic or surgical procedure table; an elongate arm adapted to be slidably mounted on said elongated element, said elongate arm having a plurality of open channels; a plurality of flexible elements adapted to be received on said elongate arm, said flexible elements having a plurality of raised portions adapted to be received by said open channels; and a gripping device connected to each of said plurality of flexible elements;
grasping the body fat folds with said gripping device connected to each of said plurality of flexible elements; and
positioning said flexible elements through said open channels of said elongate arm wherein said raised portions hold each flexible element in a temporary fixed position retracting said fat folds or excess skin folds from interfering with diagnostic or surgical procedures.

10. The method according to claim 9 wherein said raised portions define generally a circular area.

11. The method according to claim 9 wherein said elongate arm defines generally a curved area.

12. The method according to claim 9 wherein said gripping device is bifurcated.

13. The method according to claim 9 wherein said device further comprises a material covering said gripping device.

14. The method of claim 13 wherein said material is made of an elastomeric material.

15. The method of claim 13 wherein said material is replaceable.
